(19) United States
(12) Patent Application Publication
(10) Pub. No.: US 2012/0107896 A1
(43) Pub. Date: May 3, 2012

(54) Method for Treating a Biological Material Comprising Living Cells

(76) Inventors: Dirk Wandke, Heilbad Heiligenstadt (DE); Andy Kaemling, Birkenfelde (DE); Cindy Kaemling, Birkenfelde (DE); Benedikt Busse, Goettingen (DE)

(21) Appl. No.: 13/060,536

(22) PCT Filed: Sep. 2, 2009
(86) PCT No.: PCT/EP2009/006357

§ 371 Date: May 4, 2011

(30) Foreign Application Priority Data
Sep. 5, 2008 (DE) 10 2008 045 830.9

(51) Int. Cl. C12N 13/00

(52) U.S. Cl. 435/173.6

(57) ABSTRACT

A plasma containing at least one reactive species are used to deliver agents into living cells or an extracellular matrix of the living cells. The method and system allows for treatment of the living cells without causing irreversible damage to the cell membranes of the living cells. The plasma is produced by dielectric barrier discharge.

The invention relates to a method for treating a biological material comprising living cells.

Electroporation is a technique for treating a biological material comprising living cells, more particularly in order to permeabilize cell membranes in order, by way of example and in particular, to introduce DNA into cells (transformation). Electroporation is often used in molecular biology, wherein, in the areas of food process engineering and bioprocess engineering, electroporation can be used to improve mass transfer processes or to inactivate microorganisms.

An electrical field which is produced in general by a rapidly discharging capacitor produces microscopically small holes in the treated cell membrane, which reclose within milliseconds. This effect of electroporation has been known for many decades. The induction of pores causes a loss of semipermeability in the cell membrane and the release of intracellular constituents. For electroporation, use is made of what is known as an electroporator, that is to say equipment which produces an electrical field. The electroporator has in general space for a cuvette or other storage media, into which, by way of example and in particular, a cell suspension is pipetted, with the corresponding electrodes being located in the cell suspension. However, during electroporation, care has to be taken that the voltages and currents applied and therefore the outputs delivered are not so high that irreparable damage occurs at the cell membranes.

DE 10 2007 030 915 A1 discloses in particular an apparatus for treating surfaces with a plasma which is produced by means of an electrode over a solid dielectric by a dielectric-barrier gas discharge, wherein the apparatus has a flexible active surface which is directly adjacent to the plasma during the treatment.

DE 20 2006 009 481 U1 claims in particular an apparatus for treating surfaces of the human body, for example nails, the skin, or the like, with an electrode and an opposing electrode, which are both connected to a high-voltage source, wherein a plasma is produced between the electrode and the surface which is to be treated and is electrically connected to the opposing electrode, wherein the opposing electrode is formed as a support area for a body part to be treated and/or is a constituent of such a support area.

DE 601 21 356 T2 discloses in particular an apparatus for treating a skin surface of a patient, comprising: a probe having an opening in order to be in contact with the skin surface, the probe having in addition a first input port and a second input port; a radio frequency generator which provides a radio frequency voltage; a vacuum pump which provides a vacuum; a suction pipe which is connected between the vacuum pump and the probe, wherein the suction pipe provides the vacuum in the probe via the first input port; a coaxial cable which provides the radio frequency voltage in the probe via the second inport port; an electrode arranged in the probe and connected to the coaxial cable, wherein the electrode is configured to receive the radio frequency power of the generator and to provide a glow discharge when the vacuum is provided in the probe by means of the vacuum and the vacuum pump, wherein the glow discharge provides a substantially uniform heating of the skin surface down to at least a predetermined depth beneath the skin surface.

EP 0 523 961 A1 discloses in particular a cosmetic application system in which, by means of electrostatic charging, appropriately chargeable cosmetic constituents can be applied in particular to parts of the body.

DE 602 173 93 T2 discloses a method for treating a biological material comprising cells, wherein electroporation is used to deliver agents into the cells. A disadvantage here is the relatively low efficiency of introduction.

The problem addressed by the invention is therefore that of providing an efficient method of this generic type which virtually eliminates irreversible damage to the cell membranes of living cells.

This problem is solved according to the invention by a method as claimed in claim 1 and also by the use as claimed in claim 22.

In the method according to the invention for treating a biological material comprising living cells, a plasma is produced by means of a dielectric-barrier discharge, wherein, by means of this plasma and at least one reactive species, agents are at least partially delivered into part of the living cells and/or part of the extracellular matrix.

The specific characteristics of the plasma result in fields of use in the medical and cosmetic areas, in particular for application to skin or else for internal applications. The effects which can be used in this case include:

Promoting the absorbing capacity of the treated biological tissue/of the treated cells for substances and active ingredients; promoting the incorporation (deposit effect) for substances and active ingredients into the tissue to be treated/of the treated cells; promoting the microcirculation and resorption of materials/substances; locally anesthetizing action; inducing a spontaneous tissue reaction with stimulation of cellular repair mechanisms.

According to the invention, a dielectric-barrier discharge is understood to mean one in which a discharge takes place via an electrode, wherein, between the electrode and the cell area to be treated, use is made of a dielectric, preferably in the form of certain solid dielectrics, thus acting as a capacitor.

According to the invention, agents are at least partially delivered into living cells and/or the corresponding extracellular matrix with the aid of the plasma and reactive species which can be excited by the plasma—but need not be—wherein the use of plasma and the presence of at least one reactive species, for example and in particular radicals or ions, enable a temporary relaxation of the cell junctions and of the tissue mass. As a result, crossover capacity is achieved generally with a relatively commonly occurring increase in storage capacity (depot effect). This can, if the cells are stimulated, therefore lead to a temporary sublethal increase in the absorbing capacity of individual cells for agents which in turn leads to an activation of repair mechanisms. In addition to the local effect, it is also possible to apply systemically acting substances which, owing to promotion of the penetration and storage of materials into the dermis from the dermal depot, can enter the bloodstream.

The agents are those from the group consisting of peptides, hormones, hormone analogs, corticoids, immunosuppressives, vitamins, antihistamine preparations, antiphlogistics, painkillers (NSAIDs, opioids), local anesthetics, heparin preparations, antibiotics, cosmetics, colloidal care products, skin toning products, dsDNA (double-stranded), ssDNA (single-stranded), miRNA (microRNA), siRNA (small interfering RNA), shRNA (short hairpin RNA).

Advantageously, the reactive species is at least one species from the group consisting of free radicals, ions, and molecules, ions, radicals, atoms excited by the plasma, wherein the term “molecules excited by the plasma” is to be understood to mean those whose vibrational degrees of freedom are excited or are at higher vibration levels, wherein translational, bending and also rotational and torsional vibrations are included and/or at least one electron has been raised to a higher energy level.

Advantageously, the reactive species is a species from the group consisting of atomic nitrogen, atomic oxygen, noble gases, atomic hydrogen, OH-containing molecules, CH-containing molecules, CO-containing molecules, NH-containing molecules, alcohols, esters, aldehydes, ketones, amines, amides, ammonia, nitrogen oxides, halogens, such as, in particular, fluorine, chlorine, bromine, and iodine.

Advantageously, in the method according to the invention, use is made of an apparatus having a flexible active surface, that is to say one whose shape can be reversibly deformed, which surface is directly adjacent to the plasma during the treatment.

According to the invention, the term active surface means a surface of the apparatus which is directly adjacent to the plasma during the treatment—that is to say when the plasma exists—and, by virtue of the general material characteristics, the material has a dielectric constant which is not equal to zero, thus forming a dielectric barrier for the gas discharge, and therefore having a corresponding effect. The dielectric itself is solid in the aggregate state and can be, but need not be, coated with one or more materials, which, for the first time, allows a certain amount of flexibility, for example, when, although the dielectric is solid, it is, however, in the form of powder and this powder is applied to or introduced into a material similar to rubber, which has visco-elastic characteristics and can be appropriately shaped.

This allows a mechanical matching capability to the local circumstances while at the same time providing the effect of forming a dielectric barrier for a corresponding gas discharge. It is, of course, also feasible for a solid granular or powder dielectric to be located/arranged on a flexible substrate.

It is, however, also feasible to provide an intrinsically solid—in the sense of not being flexible—existing solid dielectric with a coating which as such is flexible and/or for the first time allows or improves the flexibility as such, thus providing a flexible active surface for the purposes according to the invention, with respect to the effect of the solid dielectric and its configuration.

The fundamental principle of the apparatus is based on an object to be treated being subjected to a plasma which is produced by means of an electrode and an opposing electrode, with a dielectric being advantageously arranged between the object to be treated and the electrode such that a plasma is produced by means of a dielectric-barrier gas discharge, and this plasma is then applied to the object to be treated and agents are delivered into living cells and/or an extracellular matrix. According to the invention, at least some of the agents are applied to the biological material comprising living cells before, during, or after plasma exposure.

This excitation principle results in a cold gas discharge (plasma) being formed between the electrode and the treatment area. This makes it possible to treat surfaces and/or cavities at a short distance away (0.1-50 mm), that is to say without making contact, and/or resting thereon in a locally highly confined area and/or by arranging a plurality of flexible electrodes in a row or a fabric-like structure, even over a large area with a different topology. The specific characteristics of the plasma result not only in use for treatment and disinfection of the corresponding surfaces and/or cavities, but also in fields of use in the medical area, in particular for application to skin or else for internal applications.

The effects which can be used in this case comprise, for example, low-dose UV irradiation in the useful UV-A and UV-3 wavelength band, and the reactive gas species in the gas discharge (plasma). The method therefore combines a plurality of effective effects, thus resulting in a reduction in itching, a promotion of microcirculation, an immunomodulatory effect, and a bactericidal and fungicidal effect, which is in turn highly useful for an application for at least some shoe inserts. At the same time, the apparatus can also be used for treating surfaces and/or cavities, in particular of skin, since this allows the treatment of skin diseases with accompanying intensive itching, or else the treatment of chronic lack of wound healing on the basis of microcirculation disturbances.

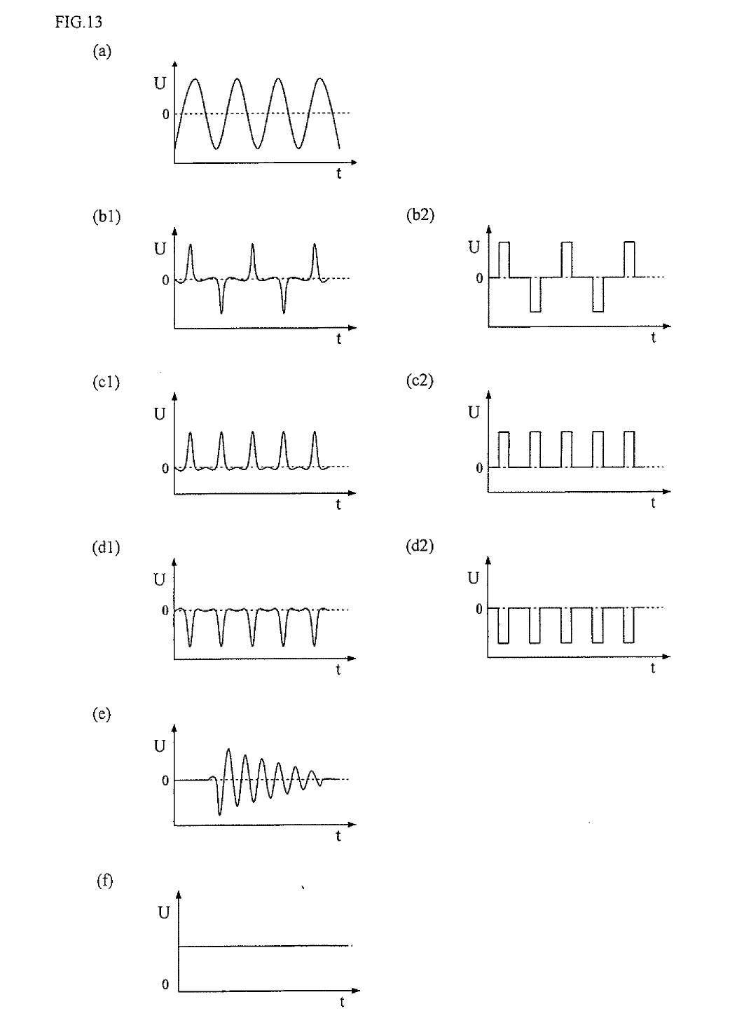

The apparatus, and the method according to the invention, use voltages in the range from 100 to 100 000 volts. The applied voltage (see FIG. 13) may be sinusoidal (a), pulsed (b1, b2, c1, c2, d1, d2) (unipolar or bipolar), may be in the form of a radio-frequency pulse (e), or may be in the form of a DC voltage (f). Combinations of different voltage forms can also be used. The electrode may be composed of electrically highly conductive materials, with the opposing electrode being composed of the same materials and/or with the object to be treated forming the opposing electrode. Normally, the solid dielectrics are composed of glasses, ceramics, or plastics.

The AC voltage frequency is normally 1 Hertz to 100 MHz. The plasma treatment application times are governed by the field of use and may extend from a few milliseconds through several minutes to a few hours.

Exemplary electrical parameters as a function of the electrode area A are:
a) A(ceramic)=0.79 cm²; U=10 kV; f(P)=385 Hz; E(discharge)=033 mJ;
b) A(ceramic)=2 cm²; U=10 kV; f(P)=385 Hz; E(discharge)=0.55 mJ;
   A=electrode area, V=applied voltage, f(P)=AC frequency for the plasma production, and E(discharge)=energy of the discharge for producing the plasma.

An advantageous embodiment is one in which the apparatus has a flexible active surface which is directly adjacent to the plasma during the treatment, particularly when the solid dielectric is equipped with a flexible surface, which, for example, can be provided by the dielectric being in the form of a granulate and/or a powder. However, this can also be achieved by the dielectric being arranged, for example as a fine powder, on and/or in a flexible hollow fiber, for example composed of glasses, ceramics or plastics, or by the dielectric itself being formed by a flexible hollow fiber. The hollow fiber may have an internal diameter of 0.5 μm to 2000 μm. The wall thicknesses are in the range from 10 μm to 2000 μm. The length of the hollow fibers and the effective active length associated with this may extend from a few millimeters to several meters. The electrical connection of a connection to the electrode or opposing electrode is ensured in particular and for example via a metallic contact at the end of the hollow fiber. By way of example and in particular, this is introduced into the hollow fiber such that it closes the hollow fiber, if necessary also in a gas-tight manner, thus allowing a conductive connection. The hollow fiber, contact, and connection are accommodated in a holder so as to allow a secure connection from the voltage supply to the contact.

Because of the flexibility of the active surface, the apparatus can even be applied in difficult situations such as cavities—for example in the case of open wounds—so as to ensure that the plasma has a uniform and homogeneous effect on the surface to be treated.

In this context, it is advantageous for the electrode to rest at least partially directly on the surface of the dielectric in order to build up as high a field strength as possible for the electrical field which is formed in the dielectric between the electrode and the opposing electrode, and when the surface of a specific object/subject to be treated, for example in the case of skin, is located between the electrode and the opposing electrode.

In this context and as an alternative embodiment, it is advantageous if the electrode is separated at least partially by means of a spacer from the surface of the dielectric. If the spacer is in this way in the form of a conductive material, and therefore not a dielectric, with the spacer being designed to have an appropriate electrical conductivity between the electrical conductivity of the electrode (very highly conductive) and the electrical conductivity of the dielectric (poorly conductive to having an insulating effect), in order in this way to homogenize the electrical field vectors, this leads to the plasma propagating better and more uniformly over an area.

In this context, it is advantageous for the electrode to rest at least partially on the dielectric, as a coating, since this results in a highly flexible embodiment, particularly when the dielectric is in the form of a flexible hollow conductor.

However, it is also feasible for the electrode to be formed from solid material, as a result of which, if the dielectric is in the form of a flexible hollow fiber, the electrode is arranged, as solid material, securely in the flexible hollow fiber.

It is also advantageous for the electrode to be in the form of a granulate and/or a powder, in order in this way to ensure the flexibility (for example capability to bend) of at least a part of the apparatus.

However, it is also feasible and advantageous if, in the operating state, the electrode is an ionized gas, thus resulting in a particularly high degree of flexibility (inter alia capability to bend) of the fiber with an appropriate configuration of the dielectric on and/or in a flexible hollow fiber, or as the hollow fiber itself, since there is no core material as a solid material.

For the application of the plasma to a surface, it is particularly advantageous for the apparatus according to the invention to have an opposing electrode since this allows the application and guidance of the plasma to be controlled better, in contrast to embodiments in which the object to be treated effectively acts as the opposing electrode.

Furthermore, it is advantageous for the apparatus according to the invention to have a gas extraction device, which in particular is flexible, and/or a gas supply device, which in particular is flexible, in order in this way to allow the plasma that is produced to be controlled specifically by means of the gas discharge, in order, for example in individual cases, to remove any undesirable oxygen radicals or nitrogen oxides as quickly as possible, and in order to specifically supply gases in order, for example, to cool the treatment area and/or to deliberately cause reactions on the surface and/or in the cavities, and/or to stabilize the plasma. The term “flexible” means, in terms of a reversibly deformable shape, the capability to align and/or place the corresponding device in order to satisfy different topical requirements. By way of example, the gas supply device and gas extraction device may in this case be formed essentially by flexible tubes.

By way of example, the gas extraction device and/or the gas supply device may also be in the form of flexible hollow fibers, since this is particularly advantageous for providing and/or improving the flexibility of the overall system.

Finally, it is advantageous if at least the one hollow fiber intrinsically or with at least one other supporting element, for example in the form of a fiber, forms an element which is fabric-like in terms of textile weaving technology, for example in the form of a nonwoven, since this nevertheless allows a relatively large surface to be treated to be treated uniformly, despite having a different topology. A fabric-like element such as this, for example in the form of a nonwoven, can be incorporated in fabrics and/or healing apparatuses such as bandages or prostheses.

The shape of the fabric may be configured as appropriate for its purpose. Possible shapes are, for example and in particular, round or polygonal. The surface of a fabric-like element such as this may have an active area of 10 mm² up to 1 m², or more.

However, it is also feasible and advantageous for flexible electrodes, in particular a flexible gas supply and/or in particular a flexible gas extraction, to be arranged such that a free plasma flame is formed. In this case, the flexible electrodes may have a dielectric barrier (shield) on one side or both sides. This embodiment makes it possible to apply a plasma to surfaces and/or cavities which are further away than the other stated embodiments (up to several cm). This embodiment works independently of the conductivity at the surface, and of its surface structure.

Furthermore, the flexible electrodes allow the plasma flame to be deflected by actuators and/or a position unit in the X and/or Y direction (chosen using any desired Cartesian system). This is particularly advantageous since this allows the plasma flame to be guided over the surface.

However, it is also feasible for there to be no need for flexibility, and so the apparatus according to the invention also has a solid (rigid) surface, for example in plate form, which is directly adjacent to the plasma during the treatment, particularly when the dielectric itself has a solid surface.

The method according to the invention can be used to enable a vector-free transfer of dsDNA, ssDNA, miRNA, siRNA, shRNA, or genes. According to the invention, the term vector is understood to mean DNA molecules which, after incorporation of foreign DNA, are used for introduction and propagation thereof in a host cell.

Possible apparatuses for carrying out the method according to the invention will be explained and described further in the following text with reference to preferred exemplary embodiments, which are illustrated in the figures, in which:

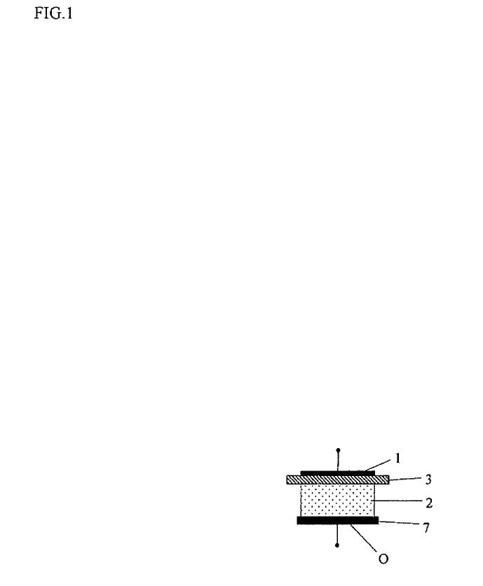

FIG. 1 shows a sketch of the functional principle of one embodiment from the prior art;

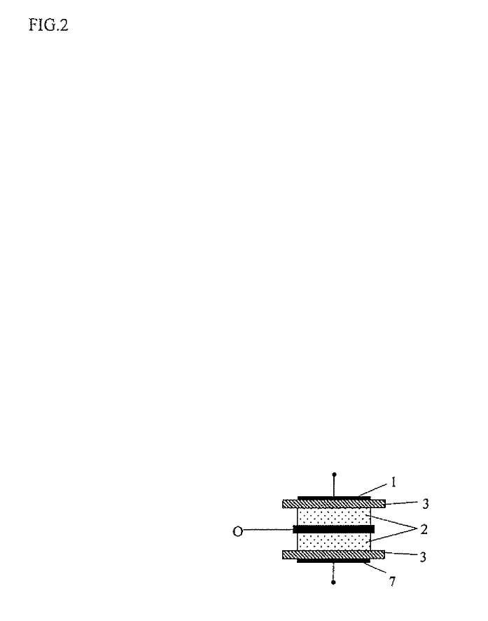

FIG. 2 shows a sketch of the functional principle of a further embodiment from the prior art;

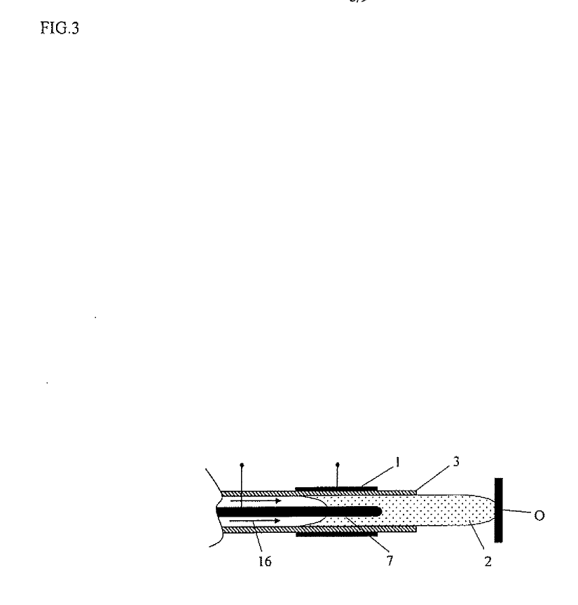

FIG. 3 shows a sketch of the functional principle of a third embodiment from the prior art;

FIG. 4 shows a sketch, in the form of a cross section, of a first embodiment according to the invention;

FIG. 5 shows a sketch, in the form of a cross section, of a second embodiment for the method according to the invention;

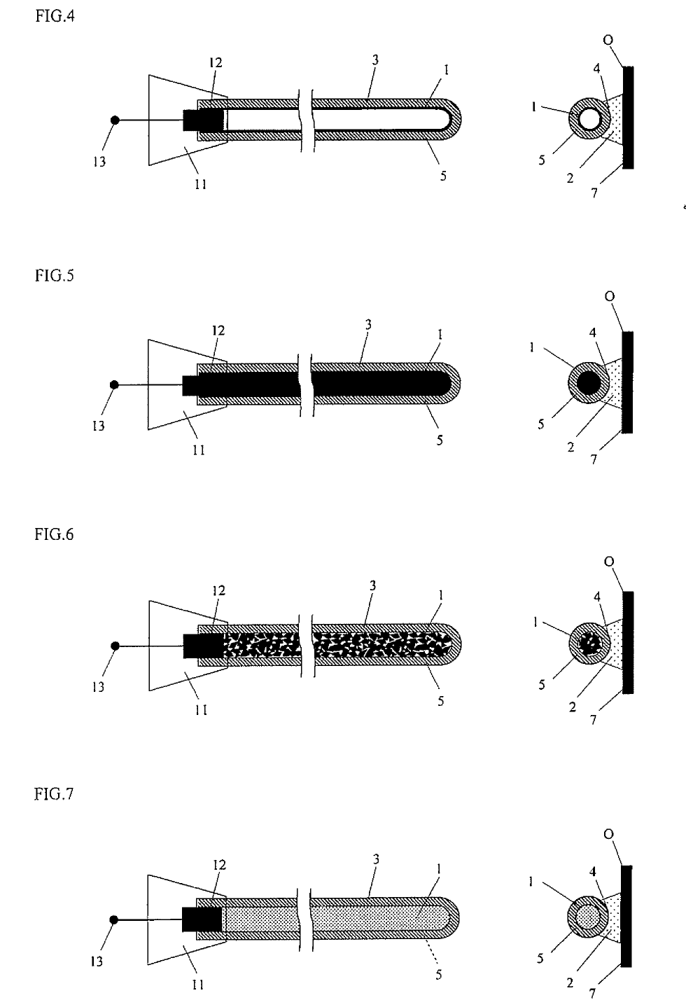

FIG. 6 shows a sketch, in the form of a cross section, of a third embodiment for the method according to the invention;

FIG. 7 shows a sketch, in the form of a cross section, of a fourth embodiment for the method according to the invention;

FIG. 8 shows a sketch, in the form of a cross section, of a fifth embodiment for the method according to the invention;

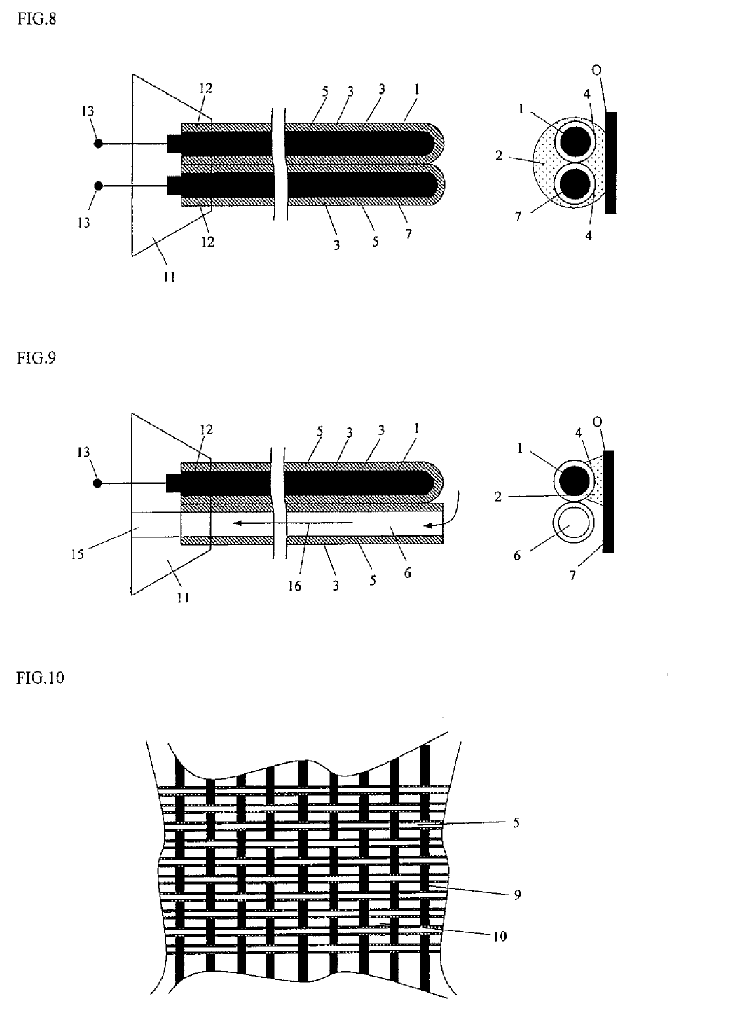

FIG. 9 shows a sketch, in the form of a cross section, of a sixth embodiment for the method according to the invention;

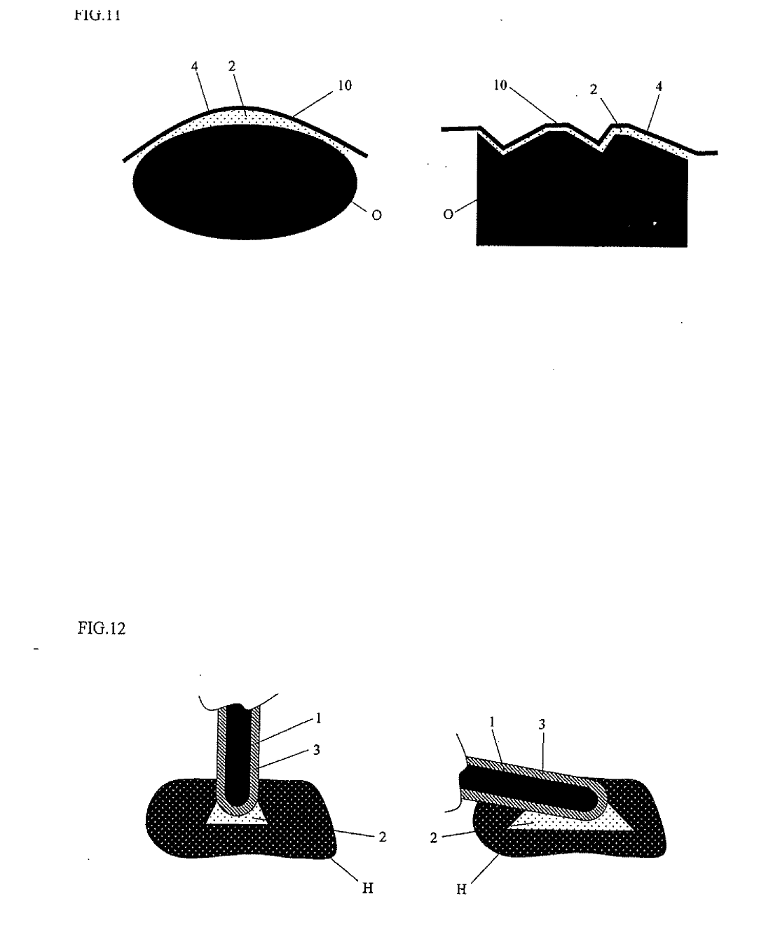

FIG. 10 shows a sketch, in the form of a cross section, of a seventh embodiment for the method according to the invention;

FIG. 11 shows a sketch, in the form of a cross section, of a medical application;

FIG. 12 shows a functional sketch of a conventional application for a method according to the invention;

FIG. 13 shows a sketch of various voltage forms which can be applied to the electrode;

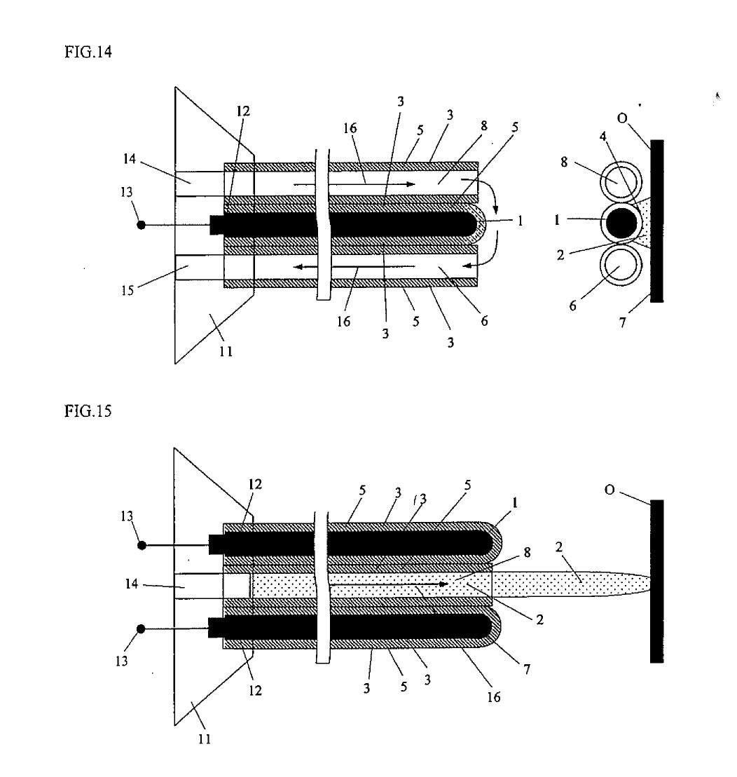

FIGS. 14-17 show sketches, in the form of cross sections, of eighth to eleventh embodiments.

FIG. 1 shows the functional layout of an apparatus for the method according to the invention—as known from the prior art—in which an electrode (1) and the object O to be examined (conductively) acting as an opposing electrode 7 produce an electrical field when an AC voltage of several thousand volts and at frequencies up to the megahertz range is applied, in which air is converted by a corresponding gas discharge to a plasma 2 between the electrodes, as a result of which the object to be treated, as the opposing electrode 7, is treated directly topically by the plasma.

The principle (prior art) illustrated in FIG. 2 differs from that disclosed in FIG. 1 only in that the object to be treated is arranged between an electrode 1 and an opposing electrode 7, and is therefore located centrally in the plasma that is produced.

As can be seen from FIG. 3 (prior art), a corresponding plasma beam 2 is produced via a gas discharge, via a tubular supply of a gas to be ionized, by means of an electrode 1 and an opposing electrode 7, and this plasma beam is aimed directly at an object to be treated.

Fundamentally, in the case of the principles illustrated in FIGS. 1 and 2, an appropriate solid dielectric is located between the electrode and the object to be treated, with an appropriate solid dielectric 3 furthermore also being provided in FIG. 2, between the opposing electrode 7 and the object to be treated.

The following figures explain examples of various embodiments according to the invention.

In FIG. 4, the dielectric material is composed of glass, ceramic, or plastic and is in the form of a flexible hollow fiber 5, with the inner wall of the hollow fiber 5 being coated with an electrically conductive material such as metals, doped semiconductors, or conductive metal-oxide layers (ITO) (indium-tin oxide), with the coating acting as the electrode 1. In a configuration such as this, the object to be treated in general acts as the opposing electrode when only one hollow fiber 5 is used.

The embodiment shown in FIG. 5 differs from that shown in FIG. 4 only in that the electrode 1 is formed from solid material, and is composed of conductive materials such as metals and/or metal alloys or the like.

The embodiment shown in FIG. 6 differs from those shown in FIGS. 4 and 5 in that the electrode 1 is in the form of a powder, composed of conductive materials such as metals and/or metal alloys or the like.

The embodiment shown in FIG. 7 differs from the previous embodiments in that the electrode is in the form of an ionized gas, for example noble gases or other inert gases, or gas mixtures thereof, or is composed of other gases which can be ionized, with the ionized gas being produced, for example, in that the gas is ionized (plasma) by the application of a high voltage that is greater than the breakdown voltage. The ionized gas is now electrically conductive and can therefore be used as an electrode.

The embodiment shown in FIG. 8 differs from those shown in FIGS. 4, 5, 6, and 7 in that two corresponding hollow fibers 5 composed of dielectric material and each having solid-material electrodes are arranged adjacent to one another in the longitudinal direction, as a result of which the upper electrode acts as the electrode 1 and the lower electrode acts as the opposing electrode 5 when an appropriate voltage is applied, in such a way that the geometric arrangement of these two hollow fibers results in a specific plasma geometry, in which case, furthermore a plurality of hollow fibers are also feasible in order to produce a corresponding plasma geometry.

FIG. 9 differs from the embodiments shown in FIGS. 4, 5, 6, and 7 in that an appropriate extraction device 6 is arranged adjacent to the hollow fiber 5 in the longitudinal direction such that any undesirable components, for example oxygen radicals that are produced, are quickly removed from the object to be treated, for example in order not to irritate sensitive skin particles.

FIG. 14 differs from the embodiments shown in FIGS. 4, 5, 6, and 7 in that an appropriate, flexible gas extraction device 6 and a flexible gas supply device 8 are arranged adjacent to the hollow fiber 5 in the longitudinal direction, such that any undesirable components, for example oxygen radicals that are produced, are quickly removed from the object to be treated, or else to specifically supply gases in order, for example, to cool the treatment area and/or to deliberately cause reactions.

As can be seen from FIG. 10, a plurality of hollow fibers 5 composed of dielectric material, or having a dielectric coating composed of glass, ceramic, or plastic, and provided with electrodes, for example in the form of an inner coating (see the embodiment in FIG. 4), in conjunction with further supporting elements 9 in the form of fibers form a fabric-like element 10, thus allowing appropriately adequate and matched shaping, and therefore application, even in the case of difficult topologies (see FIG. 11).

Finally, FIG. 12 shows a sketch of a conventional application with respect to a part of a skin area H (in this case, the skin H is the object O to be treated), acting as an opposing electrode and object.

FIGS. 15 to 17 show different embodiments, which differ from the previous embodiments in that the electrode or electrodes and/or the gas supply device are/is arranged such that a free plasma flame is formed. The free plasma flame of the plasma 2 emerging from the flexible apparatus can be used for direct topical application.

In the embodiment illustrated in FIG. 15, the plasma is provided with a dielectric barrier, by means of appropriate solid dielectrics 3, in order to prevent direct contact, with respect to the electrode 1 and the opposing electrode 7.

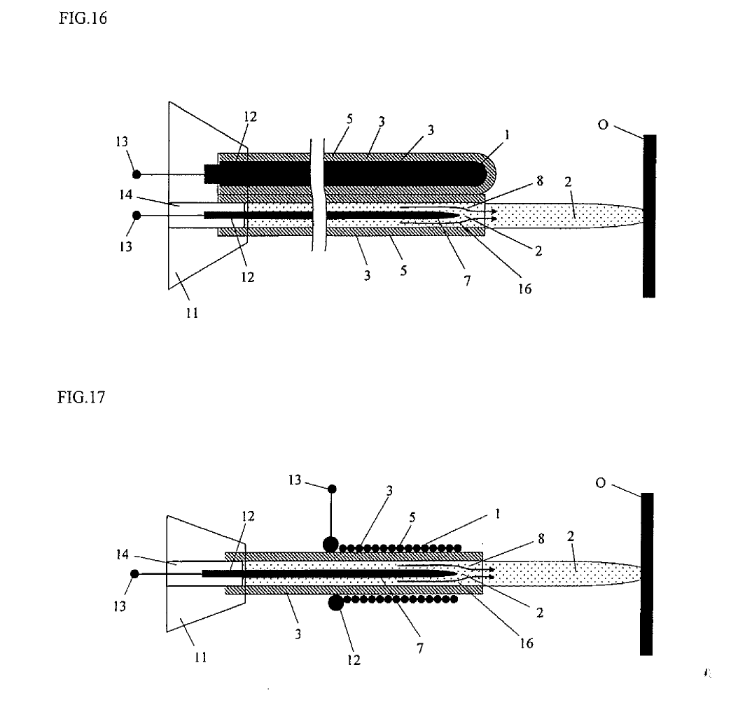

In the embodiments in FIGS. 16 and 17, only one simple dielectric barrier is provided, such that the plasma cannot make direct contact with the electrode 1 through the solid dielectric 3, but makes direct contact with the opposing electrode 7, since this is located in the plasma itself and, for example, is in the form of an electrically conductive flexible wire.

The embodiment in FIG. 17 differs essentially from that in FIG. 16 in that the electrode 1 is arranged in a spiral shape as an outer electrode around the solid dielectric (in this case: hollow-fiber material) in order to provide and/or to assist a certain amount of mechanical flexibility. It is, of course, also feasible for the embodiments shown in FIGS. 15 to 17 to be equipped with a gas extraction device as shown in FIG. 14.

### LIST OF REFERENCE SYMBOLS

O—Object
H—Skin
1—Electrode
2—Plasma
3—Solid dielectric
4—Active surface
5—Hollow fiber
6—Gas extraction device
7—Opposing electrode
8—Gas supply device
9—Supporting element
10—Fabric-like element
11—Holder
12—Contact
13—Connection, electrical
14—Gas inlet
15—Gas outlet
16—Gas flow

1. A method for treating a biological material comprising living cells, wherein, by means of a plasma and at least one reactive species and the current flow prevailing in the plasma, agents are at least partially delivered into part of the living cells and/or part of the extracellular matrix, wherein the plasma is produced by means of a dielectric-barrier discharge, wherein the agents are those from the group consisting of peptides, hormones, hormone analogs, corticoids, immunosuppressives, vitamins, antihistamine preparations, antiphlogistics, painkillers, local anesthetics, heparin preparations, antibiotics, cosmetics, colloidal care products, skin toning products, dsDNA, ssDNA, miRNA, siRNA, shRNA, and genes.

2. The method as claimed in claim 1, characterized in that the reactive species is a species from the group consisting of free radicals, ions, and molecules, ions, radicals, atoms excited by the plasma.

3. The method as claimed in claim 2, characterized in that the reactive species is a species from the group consisting of atomic nitrogen, atomic oxygen, noble gases, atomic hydrogen, OH-containing molecules, CH-containing molecules, CO-containing molecules, NH-containing molecules, alcohols, esters, aldehydes, ketones, amines, amides, ammonia, nitrogen oxides, halogens.

4. The method as claimed in claim 1, characterized in that an apparatus for treating surfaces is used, plasma (2) which is produced by means of an electrode (1) over a solid dielectric (3) by a dielectric-barrier gas discharge, wherein the apparatus has an active surface (4) which has a reversibly deformable shape and which is directly adjacent to the plasma (2) during the treatment.

5. The method as claimed in claim 4, characterized in that the dielectric (3) has a surface (4) with a reversibly deformable shape.

6. The method as claimed in claim 4, characterized in that the dielectric (3) is arranged on and/or in a flexible hollow fiber (5).

7. The method as claimed in claim 4, characterized in that the dielectric (3) is a flexible hollow fiber (5).

8. The method as claimed in claim 1, characterized in that an apparatus for treating surfaces is used, plasma (2) which is produced by means of an electrode (1) over a solid dielectric (3) by a dielectric-barrier gas discharge, wherein the apparatus has a solid active surface (4) which is directly adjacent to the plasma (2) during the treatment.

9. The method as claimed in claim 8, characterized in that the dielectric (3) has a solid surface (4).

10. The method as claimed in claim 4, characterized in that the dielectric (3) is in the form of a granulate and/or a powder.

11. The method as claimed in claim 4, characterized in that the electrode (1) rests at least partially directly on the active surface of the dielectric (3).

12. The method as claimed in claim 4, characterized in that the electrode (1) is separated at least partially by means of a spacer from the active surface of the dielectric.

13. The method as claimed in claim 11, characterized in that the electrode (1) rests at least partially on the dielectric (3), as a coating.

14. The method as claimed in claim 11, characterized in that the electrode (1) is formed from solid material.

15. The method as claimed in claim 11, characterized in that the electrode (1) is in the form of a granulate and/or a powder.

16. The method as claimed in claim 11, characterized in that, in the operating state, the electrode (1) is an ionized gas.

17. The method as claimed in claim 4, characterized in that it includes an opposing electrode (7).

18. The method as claimed in claim 4, characterized in that the apparatus it includes a gas extraction device (6) and/or a gas supply device (8).

19. The method as claimed in claim 18, characterized in that the gas extraction device (6) and/or the gas supply device (8) are/is flexible.

20. The method as claimed in claim 6, characterized in that at least the one hollow fiber (5) intrinsically or with at least one other supporting element (9) forms a fabric-like element (10).

21. The method as claimed in claim 4, characterized in that the electrode and/or the gas supply device and/or the gas extraction device are/is arranged such that a free plasma flame is formed.

22. The use of a method as claimed in claim 1 for the vector-free transfer of at least one agent from the group consisting of dsDNA, ssDNA, miRNA, siRNA, shRNA, gene.

23. The method as claimed in claim 7, characterized in that at least the one hollow fiber (5) intrinsically or with at least one other supporting element (9) forms a fabric-like element (10).

24. The method as claimed in claim 10, characterized in that the dielectric (3) is in the form of a granulate and/or a powder.

25. The method as claimed in claim 8, characterized in that the apparatus includes a gas extraction device (6) and/or a gas supply device (8).

26. The method as claimed in claim 25, characterized in that the gas extraction device (6) and/or the gas supply device (8) are/is flexible.
